(19) United States
(12) Patent Application Publication
(10) Pub. No.: US 2006/0292242 A1
(43) Pub. Date: Dec. 28, 2006

(54) Use of zeolithes for reducing the proportion of lactates and ammonium in human and animal organisms

(76) Inventors: Jakob Hraschan, VILLACH-GODERSDORF (AT); Christian Herzog, Villach-Godersdorf (AT); Antonio Lelas, Zagreb (HR)

Correspondence Address: John P White;Cooper & Dunham, 1185 Avenue of the Americas, New York, NY, 10036, US

(21) Appl. No.: 10/504,283

(22) PCT Filed: Feb. 26, 2003
(86) PCT No.: PCT/AT03/00059

§ 371 Date: Aug. 14, 2006

(30) Foreign Application Priority Data
Feb. 26, 2002 (AT) A 294/2002

(51) Int. Cl. A61K 33/38; A61K 33/12; A61K 33/06; C01B 39/00

(52) U.S. Cl. 424/618; 424/683; 424/684; 423/700

(57) ABSTRACT

The invention relates to lowering the lactate values or the ammonium values in the blood of mammals, including humans. According to the invention, ground zeolites are administered orally in the form of a consumption product or a drug. In particular, the zeolite is chosen from the group of natural zeolites such as clinoptilolite, silver zeolite, mordenite, phillipsite, and analcite, and/or from the group of synthetic zeolites such as zeolite A, zeolite W, and zeolite X.

The invention relates to lowering the proportions of lactates in humans and animals, respectively, by the, preferably oral, administration of zeolites.

The various so-called consumption products (food supplements), namely ground zeolite, in particular clinoptilolite, are known to present a general adsorptive or absorptive property and to have the capacity to neutralize buffered systems.

The inventors observed that when food supplements were administered to friends of theirs who were amateur or professional athletes, the performance capacity, endurance, and recovery after exertion were clearly improved by the ingestion of zeolites in the form of a powder and capsules.

Subsequently, scientists studying sports were contacted and, as a result, it was observed in the first user observations that the values measured during ergometry were surprisingly strongly improved in regard to the lactate content.

It is generally known that a high lactate or ammonium proportion in the organism (blood) has a negative effect on the performance capacity of humans and animals.

¹Lactates are salts of lactic acid. Lactic acid: 2-hydroxypropionic acid. Aliphatic hydroxycarboxylic acid, CH₃CH(OH)—COOH; has an asymmetric carbon atom and therefore it occurs in two optically active forms (D- and L-lactic acid). In the human body, the dextrorotatory [sic; levorotatory] L(+) lactic acid is very rapidly metabolized, while the levorotatory [sic; dextrorotatory] D(−) lactic acid is metabolized only slowly and therefore it accumulates. Fermentative D(−) lactic acid, which is produced during the lactic acid fermentation of acidic milk (from the lactic sugar) from starch and grape sugar as well as in the acidification of plant material (in silos and during the preparation of sauerkraut) with the help of lactic acid bacteria.
¹This paragraph contains incomplete sentences in the German text.]

Meat lactic acid is produced from the reserve carbohydrate in the working muscle by glycolysis (glycogen). During physical exertion, there is an increase in lactic acid level in the blood. Elevated lactate values occur if, for example, due to physical effort, a so-called oxygen deficit occurs in the blood circulation. Starting at a concentration of 2 mmol per liter, the organism shifts from metabolizing fat to metabolizing sugar. Starting at a concentration of 4 mmol per liter, albumin starts to be metabolized. The higher the heart rate, the higher the concentration of the lactates. The body uses the combustion of sugar to supply the (heart) muscles. The change in the lactate values and the ammonium values is usually connected.

The purpose of the invention is to use both natural and synthetic minerals of the group of the zeolites for absorption, adsorption, and neutralization, as well as for lowering the proportion of lactates or ammonium in humans and animals. The zeolites have been crushed in conventional mineral or stone mills, or crushed by special micronization methods, including among other methods the so-called “tribomechanical activation.”

In principle, the following zeolites are suitable for use in the invention:

Natural zeolites: clinoptilolite, silver zeolite, mordenite, phillipsite, and analcite.

Synthetic zeolites: zeolite A, zeolite W, and zeolite X.

Additional natural zeolites are presumably also suitable, but they still have to be examined to determine their properties.

The described effects are achieved by oral administration (capsules for swallowing or powders that can be ingested dry, or drunk dissolved in a liquid) and also by intravenous administration. For the preferred oral administration, food and feed products made on the basis of zeolites are also suitable, besides tablets, capsules, or powders.

Possible fields of application include, besides the pharmaceutical use and use as a food product, the food industry and agriculture, as well as the field of sports (physical strength or performance increase) for humans and animals.

Information on the Mineral Zeolite:

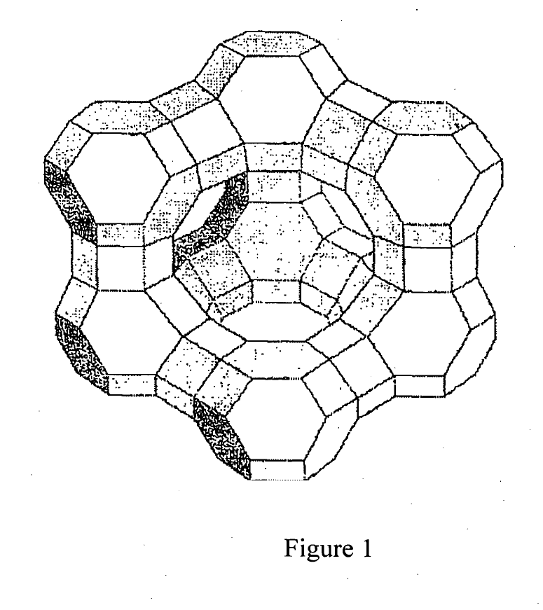

Zeolite is a crystalline substance whose structure is characterized by a crystal lattice consisting of interconnected tetrahedra, each consisting of one cation and four oxygen atoms. An example of such a structure is represented in FIG. 1. The crystal lattice contains vacancies in the form of cages and channels. The latter are usually occupied by H₂O molecules and additional cations, which can be exchanged. The channels are sufficiently large to allow the passage of host molecules. Water-rich phases can dehydrate; the dehydration usually occurs at temperatures below approximately 400° C., and is largely reversible. The crystal lattice can be interrupted by (OH,F) groups; the latter occupy the apex of a tetrahedron, which is not connected to an adjacent tetrahedron.

Structure and Properties of the Clinoptilolite:

The structure of clinoptilolite is based on a three-dimensional lattice of tetrahedra (SiO₄)⁴⁻ that are interconnected by oxygen atoms, where a part of the silicon atoms is replaced by aluminum atoms (AlO₄)⁵⁻. Partial substitution of Si⁴⁺ ions by Al³⁺ ions induces a charge deficit and it is compensated by the cations (from alkali and alkaline earth metals), which occupy certain positions in the network of channels and pores. The sizes of the channels are sufficiently large to allow the penetration into them of molecules that have a size of several tenths of one nanometer, and their absorption. The volume of these hollow channels is 24-32% of the total volume.

The properties of zeolites are derived from the special features of their crystalline structure. This special feature is that the spatial molecular structure forms channels and cavities with a constant size. In these channels, the substances can be taken up by a thick drippable fluid and gaseous aggregate state². Like the majority of silicates, the present natural substance is also inert, that is, it reacts neither with food components nor with their metabolites, nor with secretions or symbiotic metabolic products in the chemical sense. Its effects are based on physical forces (surface adhesion, electromagnetic force field of the individual crystals, Van der Waal's force, and others).
²[Possibly meaning “in the form of a thick liquid or gas.”]

Structure and Processing of the Raw Material:

The geological structure of the preferred deposits in the sedimentation layer of the flat land in East Slovakia is simple. It consists of a teolite tuff having a thickness of 90-115 m. On the surface, it outcrops over a distance of approximately 520 m. The hydrogeological conditions of the deposits are also simple. Most of the deposits carry water. A large part of the reserves can be extracted using the wall breaking [strip mining] procedure; the removal of water can occur using gravity [-driven] lines; and a small proportion of the reserves can only be extracted using shaft mining procedures.

The part responsible for the functional properties of the raw material is the zeolite mineral clinoptilolite; the average content of the deposit is 59.8 wt %. The fundamental quality parameter based on the conditions of usability is the partial ionic exchange capacity, which was determined using a 0.15M solution of NH₄Cl using the STN 72 10 76 standard (determination of the exchange capacity and of the exchange cations of argillaceous soils).

The quality of the zeolite from this deposit is determined based on the percentage content of clinoptilolite:

| % of clinoptilolite | Proportion of the raw material |
|---|---|
| 70-80 | 16.4% |
| 55-70 | 73.0% |
| 35-55 | 10.6% |

The zeolite mining is controlled qualitatively on the basis of analytical results were obtained in the context of the geological exploration of the deposit and regular evaluation of the seam prior to mining. The analytical results are recorded and plotted on the shaft maps.

The mining is carried out in accordance with the conditions of a decision to be made on the authorization of small-scale mining activity and blasting, deposit exploitation approval, the plan of the committee, preparation, and financing (POPD).

The zeolite mining in the deposit is carried out using the small-scale blast mining method. The dissolved material is loaded on trucks with an excavator, then transported to a crushing installation located directly at the deposit site. Using a truck loader, the material is led to the clay separation—coarse sorting installation, which consists of two sieves of 120 mm φ and 25 mm φ.

The sieve residue, which exceeds a size of 120 mm, is led using a gravity [-driven] line to a jaw crusher with an outlet slit of 100 mm. The raw material prepared in this manner is transported by conveyor belts into two steel containers having a capacity of 2×300 m³. The 25-120-mm fraction falls directly onto a short conveyor belt leading to the crushed material. The absorbed 0-25-mm fraction (clay, small zeolite particles) is removed, by transport with a conveyor belt system, outside of the area of the crushing installation to an uncovered mining storage facility.

The crushed zeolite is transported by truck from the crushing installation containers to a covered storage site.

Drying of the Zeolite:

The zeolite from the covered storage site is transported to the crushing line, where it is crushed using a hammer breaker to a fraction size of up to 50 mm. From the hammer crusher, zeolite is conveyed into the ground material containers for the drying lines 2 and 3. The crushed zeolite is transported from the container with a scale [chain] and rubber conveyor belt into the drying cylinder.

In the rotating drying cylinder, the material is transported further in concurrent operation with the drying medium. The inserts in the drying cylinder ensure that the material is well mixed and distributed evenly in the entire cylinder, which promotes uniform drying. The drying medium is prepared in a combustion chamber by burning the dust coal on a movable grid.

A ventilator is set up behind the electroseparator to pull the drying material,. The combustion air is driven by the ventilator under the movable grid. The temperature of the drying material in the inlet into the drying cylinder is 400-500° C. and in the outlet it is 95-105° C. The drying temperature is very important. Higher temperatures would lead to continuous destruction of the crystal structure of the clinoptilolite and to a partial loss of the application properties. The zeolite is dried, according to the standard, to an outlet humidity of 4% (less if needed). After passing the drying cylinder, the material falls into the collection ditch over a slide. Smaller material particles are pulled into a cyclone, which functions as a coarse separator. The finest fractions, which are not taken up in the cyclone, are collected by an electrofilter, where they adhere to the electrodes due to the high electric potential. The dust material, which has been collected by the cyclone and the electroseparator, is led into a container.

Grinding of the Zeolite:

For grinding the zeolite, a mill is provided. If needed, there is also the possibility of using additional mills of the same construction type and performance parameter for milling the zeolite.

The dried zeolite is conveyed from the collecting ditch under the drying lines by means of a bridge crane into a marked container, which is located above the corresponding mill, and from which the zeolite is removed using a Harding loader and led into the mill. The dosage quantity is regulated in such a manner that the performance and quality parameters of the milling are maintained. The mills are two-chamber mills. The filling of the first chamber forms milling spheres having a diameter of 30-100 mm, while the filling of the second chamber forms the short cylindrically shaped milling bodies.

The chambers are separated from each other by a separation partition, which does not sufficiently prevent the passage of dissolved material pieces into the second (remilling) chamber. The material is crushed to the desired fineness as it passes through the mill due to the impact of the milling bodies. The milled material falls from the mill through a shaking sieve and a flat closure into the container, which is set up above the Fuller pump. A ventilator aerates the mill, where the dissolved [absorbed] moisture of the input material and the heat released by the friction of the milling bodies are removed. With the Fuller pump, the milled material is conveyed through a pipeline into concrete silos.

Tests:

In a first test, 9 persons of different ages and participating in different sports were tested. Of these 9 persons, 8 produced tests that were assessable. One test subject was unable to carry out the second test, which was carried out on a treadmill, because of a foot injury. In seven of the nine test subjects, significant decreases in the lactate content of the blood could be observed within one week. This means, in particular for persons engaged in sports: a shorter recovery time, longer training units, and thus a more rapid and better training success.

Administration-dosage:

It is preferred to administer the dosage for adults orally, at a dosage of 2-30 g; if the milling result is extremely fine, it can be lowered; if it is coarser and the exertion is extreme, the dosage can also be higher. The administration can be carried out in powdered form or in gelatin capsules or similar forms, in order to ensure a protected passage through a proportion of the digestive system. The effects of these different forms of administration on the efficacy has not yet been researched. For children, dosages that have been lowered in accordance with body -weight-must be-used. No side effects or undesired effects have been noted.

Measurement Results:

The subjects, after the first of the two tests, received 2 teaspoons 3 times daily, and thus a total of approximately 12 g of the above-described zeolites, namely milled clinoptilolite, by oral administration. To compensate for any negative impact on the efficacy during the passage through the digestive tract, the subjects also received administrations of 3 gelatin capsules 3 times daily, each containing 400 mg of milled clinoptilolite (exactly the same material that was administered in powdered form), that is, they received an additional 3.6 g.

The measurement results are indicated below for each one of the subjects for two tests. Here, the weekly training of the subjects is also indicated, with the symbol “GA1” for “fundamental endurance 1” and “GA2” for “cardio or circulation load in the sugar metabolization range;” the symbols are conventional designations used in sports medicine. The diagrams show the heart rate (uppermost, highly variable curve), the lactate load (middle curve, averaged from the individual measurements plotted as small circles), and at the very bottom the speed levels during each test. From a comparison of each of the two tests, one clearly obtains the effect of the administered zeolites.

An additional test was carried out by Dr. med. Gerhard Stingl, a sports physician in Klagenfurt, Austria, on ten subjects. Here, each subject regularly received clinoptilolite for 3 months, as explained above; it was ascertained that the training and dietary habits were not changed during that period. The effect was verified using the treadmill ergometer; the lactate extraction was determined from the hyperemic right earlobe; the measurement was carried out using a biosensing lactate measuring device. The results showed a lowering of the lactate value for each subject at each load level and a significant increase in the performance capacity.

LACTATE TEST WITH REFERENCE TO MEGAMIN

| Subject 1 | Test 1 | Test 2 |
|---|---|---|
| Lactate 1 | 1.22 | 1.0 |
| Lactate 2 | 1.38 | 0.9 |
| Lactate 3 | 1.73 | 2.09 |
| Lactate 4 | 2.19 | 2.74 |
| Lactate 5 | 4.11 | 1.23 |
| Lactate 6 | | |
| Lactate 7 | | |
| Lactate 8 | | |
| Lactate 9 | | |

Training/week:
4 h ice skating (2 × 2) = GA1
4 h hip-hop (2 × 2) = GA1/2
5 h gymnastics

Personal Data

| | |
|---|---|
| Name | Test 1/2 |
| Address | |
| Address | |
| Date of birth | Feb. 6, 1965 |
| Height | 160 cm |
| Weight | 59 kg |
| Training | Hours/week |
| Resting pulse | 1/min |
| Remark | |

Examination Data

| | |
|---|---|
| Test | |
| Date | Dec. 16, 2001 |
| Starting time | 09:20:51 a.m. |
| Remark | |
| Remark | |

Calculated Values

| Desired P | 10.6 | km/h |
|---|---|---|
| P desired/kg | 00.2 | km/h/kg |
| P max | 12.0 | km/h |
| Relative performance capacity | 113% | |
| VO₂ max | 0364 | mL/min |
| VO₂ max/kg | 06.2 | mL/min/kg |

| | |
|---|---|
| HR threshold/HR max | 73% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 168 | 12 | 4.2 | 40 | 3.2 | 0 |
| 2 mmol/l | 145 | 8.5 | 2 | 28.6 | 2.2 | 0 |
| 3 mmol/l | 158 | 10.3 | 3 | 34.8 | 2.7 | 0 |
| 4 mmol/l | 166 | 11.7 | 4 | 39.1 | 3.1 | 0 |
| IAT | 123 | 6.9 | 0 | 23.7 | 1.8 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-145 | 0-8.5 | 0-28.6 | 0-2.2 | GA 1 |
| 2-3 | 154-158 | 8.5-10.3 | 28.6-34.8 | 2.2-2.7 | GA 1-2 |
| 3-4 | 158-166 | 10.3-11.7 | 34.8-39.1 | 2.7-3.1 | GA 2 |
| 4-6 | 166-168 | 11.7-11.9 | 39.1-39.8 | 3.1-3.2 | GA 2 (WSA) |

[image]

Personal Data

| | |
|---|---|
| Name | Test1/1 |
| Address | Sports teacher |
| Address | |
| Date of birth | Feb. 6, 1965 |
| Height | 160 cm |
| Weight | 59 kg |
| Training | Hours/week |
| Resting pulse | 1/min |
| Remark | |

Examination Data

| | |
|---|---|
| Test | II |
| Date | Dec. 9, 2001 |
| Starting time | 10:37:25 a.m. |
| Remark | |
| Remark | |

Calculated Values

| Desired P | 08.5 | km/h |
|---|---|---|
| P desired/kg | 00.1 | km/h/kg |
| P max | 12.0 | km/h |
| Relative performance capacity | 141% | |
| VO₂ max | 0102 | mL/min |
| VO₂ max/kg | 01.7 | mL/min/kg |

| | |
|---|---|
| HR threshold/HR max | 82% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 170 | 12 | 4.1 | 35 | 3.2 | 0 |
| 2 mmol/l | 149 | 9.4 | 2 | 26.9 | 2.5 | 0 |
| 3 mmol/l | 162 | 10.9 | 3 | 31.7 | 2.9 | 0 |
| 4 mmol/l | 169 | 11.8 | 4 | 34.5 | 3.2 | 0 |
| IAT | 140 | 8.6 | 0 | 24.2 | 2.3 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-149 | 0-9.4 | 0-26.9 | 0-2.5 | GA 1 |
| 2-3 | 149-162 | 9.4-10.9 | 26.9-31.7 | 2.5-2.9 | GA 1-2 |
| 3-4 | 162-169 | 10.9-11.8 | 31.7-34.5 | 2.9-3.2 | GA 2 |
| 4-6 | 169-169 | 11.8-11.9 | 34.5-34.8 | 3.2-3.2 | GA 2 (WSA) |

[image] LACTATE TEST WITH REFERENCE TO MEGAMIN

| Subject 2 | Test 1 | Test 2 |
|---|---|---|
| Lactate 1 | 1.14 | 0.99 |
| Lactate 2 | 1.75 | 1.03 |
| Lactate 3 | 2.45 | 2.29 |
| Lactate 4 | 3.29 | 3.08 |
| Lactate 5 | 4.19 | 4.15 |
| Lactate 6 | 6.32 | 6.25 |
| Lactate 7 | | |
| Lactate 8 | | |
| Lactate 9 | | |

Training/week:
6 h GA1
12 h GA1/2

Personal Data

| | |
|---|---|
| Name | Test 2/2 |
| Address | Skiing |
| Address | |
| Date of birth | Jul. 22, 1985 |
| Height | 163 cm |
| Weight | 56 kg |
| Training | Hours/week |
| Resting pulse | 1/min |
| Remark | |

Examination Data

| | |
|---|---|
| Test | II |
| Date | Dec. 15, 2001 |
| Starting time | 4:56:44 p.m. |
| Remark | |
| Remark | |

Calculated Values

| Desired P | 10.6 | km/h |
|---|---|---|
| P desired/kg | 00.2 | km/h/kg |
| P max | 14.0 | km/h |
| Relative performance capacity | 132% | |
| VO₂ max | 0387 | mL/min |
| VO₂ max/kg | 06.9 | mL/min/kg |

| | |
|---|---|
| HR threshold/HR max | 64% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 200 | 14 | 6.3 | 46 | 3.8 | 0 |
| 2 mmol/l | 134 | 8 | 2 | 28 | 2.1 | 0 |
| 3 mmol/l | 154 | 9.9 | 3 | 33.6 | 2.6 | 0 |
| 4 mmol/l | 173 | 11.3 | 4 | 38 | 3 | 0 |
| IAT | 127 | 7.5 | 0 | 26.1 | 1.9 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-134 | 0-8 | 0-28 | 0-2.1 | GA 1 |
| 2-3 | 134-154 | 8-9.9 | 28-33.6 | 2.1-2.6 | GA 1-2 |
| 3-4 | 154-173 | 9.9-11.3 | 33.6-38 | 2.6-3 | GA 2 |
| 4-6 | 173-197 | 11.3-13.7 | 38-45.1 | 3-3.7 | GA 2 (WSA) |

[image]

Personal Data

| | |
|---|---|
| Name | Test 2/1 |
| Address | Skiing |
| Address | |
| Date of birth | Jul. 22, 1985 |
| Height | 163 cm |
| Weight | 56 kg |
| Training | Hours/week |
| Resting pulse | 1/min |
| Remark | |

Examination Data

| | |
|---|---|
| Test | II |
| Date | Dec. 9, 2001 |
| Starting time | 4:56:44 p.m. |
| Remark | |
| Remark | |

Calculated Values

| Desired P | 10.6 | km/h |
|---|---|---|
| P desired/kg | 00.2 | km/h/kg |
| P max | 14.0 | km/h |
| Relative performance capacity | 132% | |
| VO₂ max | 0387 | mL/min |
| VO₂ max/kg | 06.9 | mL/min/kg |

| | |
|---|---|
| HR threshold/HR max | 62% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 192 | 14 | 6.3 | 46 | 3.8 | 0 |
| 2 mmol/l | 129 | 7.8 | 2 | 27.3 | 2 | 0 |
| 3 mmol/l | 152 | 9.7 | 3 | 33 | 2.5 | 0 |
| 4 mmol/l | 172 | 11.2 | 4 | 37.7 | 3 | 0 |
| IAT | 120 | 7.4 | 0 | 25.9 | 1.9 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-129 | 0-7.8 | 0-27.3 | 0-2 | GA 1 |
| 2-3 | 129-152 | 7.8-9.7 | 27.3-33 | 2-2.5 | GA 1-2 |
| 3-4 | 152-172 | 9.7-11.2 | 33-37.7 | 2.5-3 | GA 2 |
| 4-6 | 172-190 | 11.2-13.6 | 37.7-44.8 | 3-3.7 | GA 2 (WSA) |

[image] LACTATE TEST WITH REFERENCE TO MEGAMIN

| Subject 3 | Test 1 | Test 2 |
|---|---|---|
| Lactate 1 | 0.8 | 0.76 |
| Lactate 2 | 0.89 | 0.70 |
| Lactate 3 | 1.92 | 1.24 |
| Lactate 4 | 2.11 | 1.58 |
| Lactate 5 | 2.31 | 1.73 |
| Lactate 6 | 3.1 | 2.38 |
| Lactate 7 | | |
| Lactate 8 | | |
| Lactate 9 | | |

Training/week:
2 h GA1
1 h GA1/2
Snow 4 days 4 hours each

Personal Data

| | |
|---|---|
| Name | Test 3/1 |
| Address | snowboard |
| Address | |
| Date of birth | Jan. 31, 1961 |
| Height | 183 cm |
| Weight | 76 kg |
| Training | Hours/week |
| Resting pulse | 1/min |
| Remark | |

Examination Data

| | |
|---|---|
| Test | II |
| Date | Dec. 8, 2001 |
| Starting time | 1:24:36 p.m. |
| Remark | |
| Remark | |

Calculated Values

| Desired P | 10.6 | km/h |
|---|---|---|
| P desired/kg | 00.1 | km/h/kg |
| P max | 14.0 | km/h |
| Relative performance capacity | 132% | |
| VO₂ max | 0387 | mL/min |
| VO₂ max/kg | 05.1 | mL/min/kg |

| | |
|---|---|
| HR threshold/HR max | 00% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 145 | 14 | 3.1 | 14 | 14 | 0 |
| 2 mmol/l | 123 | 9.9 | 2 | 9.9 | 9.9 | 0 |
| 3 mmol/l | 143 | 13.6 | 3 | 13.6 | 13.6 | 0 |
| 4 mmol/l | 145 | 14 | 4 | 14 | 14 | 0 |

Training Table

| Lactate | HR | v | v | v | |
|---|---|---|---|---|---|
| mmol/l | 1/min | km/h | km/h | km/h | |
| 0-1 | 0-83 | 0-6.3 | 0-6.3 | 0-6.3 | REKOM |
| 1-2 | 83-123 | 6.3-9.9 | 6.3-9.9 | 6.3-9.9 | GA 1 |
| 2-3 | 123-143 | 9.9-13.6 | 9.9-13.6 | 9.9-13.6 | GA 1-2 |
| 3-4 | 143-145 | 13.6-14 | 13.6-14 | 13.6-14 | GA 2 |
| 4-6 | 145-145 | 14-14 | 14-14 | 14-14 | GA 2 (WSA) |

[image]

Personal Data

| | |
|---|---|
| Name | Test 3/2 |
| Address | |
| Address | |
| Date of birth | Jan. 31, 2001 |
| Height | 183 cm |
| Weight | 76 kg |
| Training | Hours/week |
| Resting pulse | 1/min |
| Remark | |

Examination Data

| | |
|---|---|
| Test | II |
| Date | Dec. 15, 2001 |
| Starting time | 6:41:26 p.m. |
| Remark | |
| Remark | |

Calculated Values

| Desired P | 10.6 | km/h |
|---|---|---|
| P desired/kg | 00.1 | km/h/kg |
| P max | 14.0 | km/h |
| Relative performance capacity | 132% | |
| VO₂ max | 0387 | mL/min |
| VO₂ max/kg | 05.1 | mL/min/kg |

| | |
|---|---|
| HR threshold/HR max | 00% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 138 | 14 | 2.3 | 45 | 3.8 | 0 |
| 2 mmol/l | 130 | 12.6 | 2 | 40.9 | 3.4 | 0 |
| 3 mmol/l | 138 | 14 | 3 | 45 | 3.8 | 0 |
| 4 mmol/l | 138 | 14 | 4 | 45 | 3.8 | 0 |
| IAT | 0 | 0 | 0 | 0 | 0 | 0 |

Training Table

| Lactate | HR | v | VO₂ | | |
|---|---|---|---|---|---|
| mmol/l | 1/min | km/h | ml/min/kg | Watt/kg | |
| 0-1 | 0-90 | 0-7.1 | 0-23.3 | 0-1.8 | REKOM |
| 1-2 | 90-130 | 7.1-12.6 | 23.3-40.9 | 1.8-3.4 | GA 1 |
| 2-3 | 130-138 | 12.6-14 | 40.9-45 | 3.4-3.8 | GA 1-2 |
| 3-4 | 138-138 | 14-14 | 45-45 | 3.8-3.8 | GA 2 |
| 4-6 | 138-138 | 14-14 | 45-45 | 3.8-3.8 | GA 2 (WSA) |

[image] LACTATE TEST WITH REFERENCE TO MEGAMIN

| Subject 4 | Test 1 | Test 2 |
|---|---|---|
| Lactate 1 | 0.88 | 0.79 |
| Lactate 2 | 1.51 | 0.84 |
| Lactate 3 | 1.61 | 0.94 |
| Lactate 4 | 2.27 | 1.57 |
| Lactate 5 | 3.27 | 2.62 |
| Lactate 6 | | |
| Lactate 7 | | |
| Lactate 8 | | |
| Lactate 9 | | |

Training/week:
2 × 2 h GA1
2 × 41 min GA1/2
First day severe diarrhea
Sensation of fullness [bloating]

Personal Data

| | |
|---|---|
| Name | Test 4/1 |
| Address | Amateur athlete |
| Address | |
| Date of birth | Nov. 28, 1966 |
| Height | 178 cm |
| Weight | 77 kg |
| Training | Hours/week |
| Resting pulse | 1/min |
| Remark | |

Examination Data

| | |
|---|---|
| Test | II |
| Date | Dec. 9, 2001 |
| Starting time | 09:30:00 a.m. |
| Remark | Circulation |
| Remark | |

Calculated Values

| Desired P | 10.6 | km/h |
|---|---|---|
| P desired/kg | 00.1 | km/h/kg |
| P max | 12.0 | km/h |
| Relative performance capacity | 113% | |
| VO₂ max | 0364 | mL/min |
| VO₂ max/kg | 04.7 | mL/min/kg |

| | |
|---|---|
| HR threshold/HR max | 00% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 152 | 12 | 3.3 | 39 | 3.2 | 0 |
| 2 mmol/l | 128 | 8.9 | 2 | 29.3 | 2.3 | 0 |
| 3 mmol/l | 148 | 11.4 | 3 | 37.1 | 3 | 0 |
| 4 mmol/l | 151 | 11.9 | 4 | 38.8 | 3.2 | 0 |
| IAT | 0 | 0 | 0 | 0 | 0 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-128 | 0-8.9 | 0-29.3 | 0-2.3 | GA 1 |
| 2-3 | 128-148 | 8.9-11.4 | 29.3-37.1 | 2.3-3 | GA 1-2 |
| 3-4 | 148-151 | 11.4-11.9 | 37.1-38.8 | 3-3.2 | GA 2 |
| 4-6 | 151-151 | 11.9-11.9 | 38.8-38.8 | 3.2-3.2 | GA 2 (WSA) |

[image]

[image]

Personal Data

| | |
|---|---|
| Name | TestII 4/2 |
| Address | |
| Address | |
| Date of birth | Nov. 28, 1966 |
| Height | 178 cm |
| Weight | 77 kg |
| Training | Hours/week |
| Resting pulse | 1/min |
| Remark | |

Examination Data

| | |
|---|---|
| Test | |
| Date | Dec. 16, 2001 |
| Starting time | 7:20:41 a.m. |
| Remark | |
| Remark | |

Calculated Values

| Desired P | 10.6 | km/h |
|---|---|---|
| P desired/kg | 00.1 | km/h/kg |
| P max | 12.0 | km/h |
| Relative performance capacity | 113% | |
| VO₂ max | 0364 | mL/min |
| VO₂ max/kg | 04.7 | mL/min/kg |

| | |
|---|---|
| HR threshold/HR max | 77% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 153 | 12 | 2.6 | 39 | 3.2 | 0 |
| 2 mmol/l | 141 | 11.1 | 2 | 36.2 | 2.9 | 0 |
| 3 mmol/l | 152 | 11.9 | 3 | 38.8 | 3.2 | 0 |
| 4 mmol/l | 152 | 11.9 | 4 | 38.8 | 3.2 | 0 |
| IAT | 117 | 8.6 | 0 | 28.2 | 2.3 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-115 | 0-8.2 | 0-26.8 | 0-2.2 | REKOM |
| 1-2 | 115-141 | 8.2-11.1 | 26.8-36.2 | 2.2-2.9 | GA 1 |
| 2-3 | 141-152 | 11.1-11.9 | 36.2-38.8 | 2.9-3.2 | GA 1-2 |
| 3-4 | 152-152 | 11.9-11.9 | 38.8-38.8 | 3.2-3.2 | GA 2 |
| 4-6 | 152-152 | 11.9-11.9 | 38.8-38.8 | 3.2-3.2 | GA 2 (WSA) |

LACTATE TEST WITH REFERENCE TO MEGAMIN

| Subject 5 | Test 1 | Test 2 |
|---|---|---|
| Lactate 1 | 1.62 | 1.60 |
| Lactate 2 | 1.63 | 1.76 |
| Lactate 3 | 1.69 | 1.85 |
| Lactate 4 | 1.76 | 2.19 |
| Lactate 5 | 2.67 | 2.52 |
| Lactate 6 | | |
| Lactate 7 | | |
| Lactate 8 | | |
| Lactate 9 | | |

Training/week:
None No training, too cold
Sleep disorder

Personal Data

| | |
|---|---|
| Name | Test 5/1 |
| Address | Golf retired woman |
| Address | |
| Date of birth | Jan. 11, 1935 |
| Height | 167 cm |
| Weight | 67 kg |
| Training | Hours/week |
| Resting pulse | 1/min |
| Remark | |

Examination Data

| | |
|---|---|
| Test | II |
| Date | Dec. 9, 2001 |
| Starting time | 12:34:39 p.m. |
| Remark | |
| Remark | |

Calculated Values

| Desired P | 08.5 | km/h |
|---|---|---|
| P desired/kg | 00.1 | km/h/kg |
| P max | 07.0 | km/h |
| Relative performance capacity | 82% | |
| VO₂ max | 0047 | mL/min |
| VO₂ max/kg | 00.7 | mL/min/kg |

| | |
|---|---|
| HR threshold/HR max | 96% |
| Threshold = IAT | |

Thresholds and Maximum Values

| | HR | v | Lactate | VO₂ | P | |
|---|---|---|---|---|---|---|
| | 1/min | Km/h | mmol/l | ml/min/kg | Watt/kg | |
| Maximum | 109 | 7 | 2.7 | 19 | 1.8 | 0 |
| 2 mmol/l | 100 | 6.5 | 2 | 17.4 | 1.6 | 0 |
| 3 mmol/l | 89 | 7 | 3 | 18.9 | 1.8 | 0 |
| 4 mmol/l | 89 | 7 | 4 | 18.9 | 1.8 | 0 |
| IAT | 105 | 5.5 | 0 | 14.6 | 1.4 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-100 | 0-6.5 | 0-17.4 | 0-1.6 | GA 1 |
| 2-3 | 100-89 | 6.5-7 | 17.4-18.9 | 1.6-1.8 | GA 1-2 |
| 3-4 | 89-89 | 7-7 | 18.9-18.9 | 1.8-1.8 | GA 2 |
| 4-6 | 89-89 | 7-7 | 18.9-18.9 | 1.8-1.8 | GA 2 (WSA) |

[image]

Personal Data

| | |
|---|---|
| Name | TestII 5/2 |
| Address | |
| Address | |
| Date of birth | Jan. 6, 1935 |
| Height | 167 cm |
| Weight | 67 kg |
| Training | Hours/week |
| Resting pulse | 1/min |
| Remark | |

Examination Data

| | |
|---|---|
| Test | |
| Date | Dec. 15, 2001 |
| Starting time | 09:49:07 a.m. |
| Remark | |
| Remark | |

Calculated Values

| Desired P | 10.6 | km/h |
|---|---|---|
| P desired/kg | 00.2 | km/h/kg |
| P max | 07.0 | km/h |
| Relative performance capacity | 66% | |
| VO₂ max | 0308 | mL/min |
| VO₂ max/kg | 04.6 | mL/min/kg |

| | |
|---|---|
| HR threshold/HR max | 00% |
| Threshold = IAT | |

Thresholds and Maximum Values

| | HR | v | Lactate | VO₂ | P | |
|---|---|---|---|---|---|---|
| | 1/min | km/h | mmol/l | ml/min/kg | Watt/kg | |
| Maximum | 112 | 7 | 2.5 | 24 | 1.8 | 0 |
| 2 mmol/l | 103 | 5.8 | 2 | 19.5 | 105 | 0 |
| 3 mmol/l | 0 | 0 | 3 | 0 | 0 | 0 |
| 4 mmol/l | 0 | 0 | 4 | 0 | 0 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-103 | 0-5.8 | 0-19.5 | 0-1.5 | GA 1 |
| 2-3 | 103-0 | 5.8-0 | 19.5-0 | 1.5-0 | GA 1-2 |
| 3-4 | 0-0 | 0-0 | 0-0 | 0-0 | GA 2 |
| 4-6 | 0-0 | 0-0 | 0-0 | 0-0 | GA 2 (WSA) |

[image] LACTATE TEST WITH REFERENCE TO MEGAMIN

| Subject 6 | Test 1 | Test 2 |
|---|---|---|
| Lactate 1 | 1.11 | 1.07 |
| Lactate 2 | 1.41 | 1.34 |
| Lactate 3 | 1.55 | 1.44 |
| Lactate 4 | 2.3 | 1.62 |
| Lactate 5 | 4.11 | 3.32 |
| Lactate 6 | 7.68 | 6.24 |
| Lactate 7 | 11.41 | 10.67 |
| Lactate 8 | | |
| Lactate 9 | | |

Training/week:
3 × GA1 90 min
2 × GA 1/2 40 min

Personal Data

| | |
|---|---|
| Name | Test 6/1 |
| Address | |
| Address | |
| Date of birth | Sep. 28, 1952 |
| Height | 172 cm |
| Weight | 74 kg |
| Training | Hours/week |
| Resting pulse | 1/min |
| Remark | |

Examination Data

| | |
|---|---|
| Test | II |
| Date | Dec. 3, 2001 |
| Starting time | 1:00:09 p.m. |
| Remark | |
| Remark | |

Calculated Values

| Desired P | 10.6 | km/h |
|---|---|---|
| P desired/kg | 00.1 | km/h/kg |
| P max | 17.5 | km/h |
| Relative performance capacity | 164% | |
| VO₂ max | 0426 | mL/min |
| VO₂ max/kg | 05.8 | mL/min/kg |

| | |
|---|---|
| HR threshold/HR max | 86% |
| Threshold = IAT | |

Thresholds and Maximum Values

| | HR | v | Lactate | VO₂ | v | v |
|---|---|---|---|---|---|---|
| | 1/min | km/h | mmol/l | ml/min/kg | min/kg | m/s |
| Maximum | 185 | 17.5 | 11.4 | 56 | 3.4 | 4.9 |
| 2 mmol/l | 135 | 11.7 | 2 | 38 | 5.2 | 3.2 |
| 3 mmol/l | 150 | 13 | 3 | 42 | 4.6 | 3.6 |
| 4 mmol/l | 160 | 13.9 | 4 | 44.7 | 4.3 | 3.9 |
| IAT | 159 | 13.8 | 0 | 44.5 | 4.3 | 3.8 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-135 | 0-11.7 | 0-38 | 0-5.2 | GA 1 |
| 2-3 | 135-150 | 11.7-13 | 38-42 | 5.2-4.6 | GA 1-2 |
| 3-4 | 150-160 | 13-13.9 | 42-44.7 | 4.6-4.3 | GA 2 |
| 4-6 | 160-170 | 13.9-15.2 | 44.7-49 | 4.3-4 | GA 2 (WSA) |

[image]

Personal Data

| | |
|---|---|
| Name | Test 6/2 |
| Address | triathlon |
| Address | |
| Date of birth | Sep. 28, 1952 |
| Height | 172 cm |
| Weight | 72 kg |
| Training | Hours/week |
| Resting pulse | 1/min |
| Remark | |

Examination Data

| | |
|---|---|
| Test | II |
| Date | Dec. 10, 2001 |
| Starting time | 3:00:00 p.m. |
| Remark | |
| Remark | |

Calculated Values

| Desired P | 10.6 | km/h |
|---|---|---|
| P desired/kg | 00.1 | km/h/kg |
| P max | 18.0 | km/h |
| Relative performance capacity | 169% | |
| VO₂ max | 0432 | mL/min |
| VO₂ max/kg | 06.0 | mL/min/kg |

| | |
|---|---|
| HR threshold/HR max | 91% |
| Threshold = IAT | |

Thresholds and Maximum Values

| | HR | v | Lactate | P | VO2 | |
|---|---|---|---|---|---|---|
| | 1/min | Km/h | mmol/l | Watt/kg | ml/min/kg | |
| Maximum | 185 | 18 | 10.7 | 58 | 1.9 | 0 |
| 2 mmol/l | 149 | 12.3 | 2 | 40.1 | 3.3 | 0 |
| 3 mmol/l | 164 | 13.7 | 3 | 45.1 | 3.7 | 0 |
| 4 mmol/l | 170 | 14.6 | 4 | 47.7 | 3.9 | 0 |
| IAT | 169 | 14.4 | 0 | 47.1 | 3.9 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-149 | 0-12.3 | 0-40.1 | 0-3.3 | GA 1 |
| 2-3 | 149-164 | 12.3-13.7 | 40.1-45.1 | 3.3-3.7 | GA 1-2 |
| 3-4 | 164-170 | 13.7-14.6 | 45.1-47.7 | 3.7-3.9 | GA 2 |
| 4-6 | 170-179 | 14.6-16 | 47.7-52 | 3.9-4.3 | GA 2 (WSA) |

[image] LACTATE TEST WITH REFERENCE TO MEGAMIN

| Subject 7 | Test 1 | Test 2 |
|---|---|---|
| Lactate 1 | 1.11 | 0.80 |
| Lactate 2 | 1.59 | 1.07 |
| Lactate 3 | 1.51 | 1.14 |
| Lactate 4 | 2.16 | 1.84 |
| Lactate 5 | 4.64 | 3.64 |
| Lactate 6 | 7.4 | 7.3 |
| Lactate 7 | | |
| Lactate 8 | | |
| Lactate 9 | | |

Training/week:
3 × 90 min GA 1
1 × 60 min GA 1/2

Personal Data

| | |
|---|---|
| Name | Test 7/1 |
| Address | Soccer player |
| Address | |
| Date of birth | Dec. 17, 1973 |
| Height | 183 cm |
| Weight | 72 kg |
| Training | Hours/week |
| Resting pulse | 1/min |
| Remark | |

Examination Data

| | |
|---|---|
| Test | II |
| Date | Dec. 7, 2001 |
| Starting time | 09:41:15 a.m. |
| Remark | |
| Remark | |

Calculated Values

| Desired P | 10.6 | km/h |
|---|---|---|
| P desired/kg | 00.1 | km/h/kg |
| P max | 16.0 | km/h |
| Relative performance capacity | 150% | |
| VO₂ max | 0409 | mL/min |
| VO₂ max/kg | 05.7 | mL/min/kg |

| | |
|---|---|
| HR threshold/HR max | 89% |
| Threshold = IAT | |

Thresholds and Maximum Values

| | HR | v | Lactate | P | VO₂ | |
|---|---|---|---|---|---|---|
| | 1/min | Km/h | mmol/l | Watt/kg | ml/min/kg | |
| Maximum | 186 | 16 | 7.4 | 4.3 | 52 | 0 |
| 2 mmol/l | 159 | 11.5 | 2 | 3 | 37.4 | 0 |
| 3 mmol/l | 169 | 12.8 | 3 | 3.5 | 41.9 | 0 |
| 4 mmol/l | 176 | 13.8 | 4 | 3.7 | 45.4 | 0 |
| IAT | 166 | 12.3 | 0 | 3.3 | 40.2 | 0 |

Training Table

| Lactate | HR | v | P | VO₂ | |
|---|---|---|---|---|---|
| mmol/l | 1/min | km/h | Watt/kg | ml/min/kg | |
| 0-1 | 0-0 | 0-0 | 0-0 | 0-0 | REKOM |
| 1-2 | 0-159 | 0-11.5 | 0-3 | 0-37.4 | GA 1 |
| 2-3 | 159-169 | 11.5-12.8 | 3-3.5 | 37.4-41.9 | GA 1-2 |
| 3-4 | 169-176 | 12.8-13.8 | 3.5-3.7 | 41.9-45.4 | GA 2 |
| 4-6 | 176-182 | 13.8-15.2 | 3.7-4.1 | 45.4-49.6 | GA 2 (WSA) |

[image]

Personal Data

| | |
|---|---|
| Name | Test 7/2 |
| Address | Soccer player |
| Address | |
| Date of birth | Dec. 17, 1973 |
| Height | 183 cm |
| Weight | 74 kg |
| Training | Hours/week |
| Resting pulse | 1/min |
| Remark | |

Examination Data

| | |
|---|---|
| Test | II |
| Date | Dec. 14, 2001 |
| Starting time | 12:25:17 p.m. |
| Remark | |
| Remark | |

Calculated Values

| Desired P | 10.6 | km/h |
|---|---|---|
| P desired/kg | 00.1 | km/h/kg |
| P max | 16.0 | km/h |
| Relative performance capacity | 150% | |
| VO₂ max | 0409 | mL/min |
| VO₂ max/kg | 05.5 | mL/min/kg |

| | |
|---|---|
| HR threshold/HR max | 90% |
| Threshold = IAT | |

Thresholds and Maximum Values

| | HR | v | Lactate | P | VO₂ | |
|---|---|---|---|---|---|---|
| | 1/min | km/h | mmol/l | Watt/kg | ml/min/kg | |
| Maximum | 183 | 16 | 7.3 | 4.3 | 52 | 0 |
| 2 mmol/l | 160 | 12.2 | 2 | 3.3 | 39.7 | 0 |
| 3 mmol/l | 169 | 13.4 | 3 | 3.6 | 43.3 | 0 |
| 4 mmol/l | 175 | 14.2 | 4 | 3.9 | 45.8 | 0 |
| IAT | 165 | 12.9 | 0 | 3.5 | 41.8 | 0 |

Training Table

| Lactate | HR | v | P | VO₂ | |
|---|---|---|---|---|---|
| mmol/l | 1/min | km/h | Watt/kg | ml/min/kg | |
| 0-1 | 0-139 | 0-0 | 0-2.6 | 0-32.6 | REKOM |
| 1-2 | 139-160 | 9.9-12.2 | 2.6-3.3 | 32.6-39.7 | GA 1 |
| 2-3 | 160-169 | 12.2-13.4 | 3.3-3.6 | 39.7-43.3 | GA 1-2 |
| 3-4 | 169-175 | 13.4-14.2 | 3.6-3.9 | 43.3-45.8 | GA 2 |
| 4-6 | 175-180 | 14.2-15.4 | 3.9-4.2 | 45.8-49.9 | GA 2 (WSA) |

[image] LACTATE TEST WITH REFERENCE TO MEGAMIN

| Subject 8 | Test 1 | Test 2 |
|---|---|---|
| Lactate 1 | 1.06 | 0.93 |
| Lactate 2 | 0.87 | 0.74 |
| Lactate 3 | 0.68 | 0.6 |
| Lactate 4 | 0.87 | 0.73 |
| Lactate 5 | 1.52 | 1.24 |
| Lactate 6 | 3.01 | 2.21 |
| Lactate 7 | 5.99 | 5.12 |
| Lactate 8 | 7.89 | 6.98 |
| Lactate 9 | | |

Training/week:
min 11 h
First test, slightly cooled [not certain if this refers to air temperature or body termperture.]

Personal Data

| | |
|---|---|
| Name | Test 8/1 |
| Address | Marathon |
| Address | |
| Date of birth | Jul. 28, 1971 |
| Height | 183 cm |
| Weight | 75 kg |
| Training | Hours/week |
| Resting pulse | 1/min |
| Remark | |

Examination Data

| | |
|---|---|
| Test | II |
| Date | Dec. 4, 2001 |
| Starting time | 3:55:08 p.m. |
| Remark | |
| Remark | |

Calculated Values

| Desired P | 10.6 | km/h |
|---|---|---|
| P desired/kg | 00.1 | km/h/kg |
| P max | 19.2 | km/h |
| Relative performance capacity | 180% | |
| VO₂ max | 0445 | mL/min |
| VO₂ max/kg | 05.9 | mL/min/kg |

| | |
|---|---|
| HR threshold/HR max | 90% |
| Threshold = IAT | |

Thresholds and Maximum Values

| | HR | v | Lactate | VO₂ | P | 0 |
|---|---|---|---|---|---|---|
| | 1/min | Km/h | mmol/l | ml/min/kg | Watt/kg | 0 |
| Maximum | 199 | 19.2 | 7.9 | 62 | 5.2 | 0 |
| 2 mmol/l | 173 | 14.6 | 2 | 47.3 | 4 | 0 |
| 3 mmol/l | 180 | 16.1 | 3 | 52.2 | 4.3 | 0 |
| 4 mmol/l | 186 | 17 | 4 | 54.9 | 4.6 | 0 |
| IAT | 180 | 16 | 0 | 51.9 | 4.3 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-0 | 0-0 | 0-0 | 0-0 | REKOM |
| 1-2 | 0-173 | 0-14.6 | 0-47.3 | 0-4 | GA 1 |
| 2-3 | 173-180 | 14.6-16.1 | 47.3-52.2 | 4-4.3 | GA 1-2 |
| 3-4 | 180-186 | 16.1-17 | 52.2-54.9 | 4.3-4.6 | GA 2 |
| 4-6 | 186-193 | 17-18.1 | 54.9-58.5 | 4.6-4.9 | GA 2 (WSA) |

[image]

Personal Data

| | |
|---|---|
| Name | Test 8/2 |
| Address | |
| Address | |
| Date of birth | Jul. 28, 1971 |
| Height | 183 cm |
| Weight | 75 kg |
| Training | Hours/week |
| Resting pulse | 1/min |
| Remark | |

Examination Data

| | |
|---|---|
| Test | II |
| Date | Dec. 11, 2001 |
| Starting time | 5:39:59 p.m. |
| Remark | |
| Remark | |

Calculated Values

| Desired P | 10.6 | km/h |
|---|---|---|
| P desired/kg | 00.1 | km/h/kg |
| P max | 19.5 | km/h |
| Relative performance capacity | 183% | |
| VO₂ max | 0448 | mL/min |
| VO₂ max/kg | 06.0 | mL/min/kg |

| | |
|---|---|
| HR threshold/HR max | 84% |
| Threshold = IAT | |

Thresholds and Maximum Values

| | HR | v | Lactate | VO₂ | P | |
|---|---|---|---|---|---|---|
| | 1/min | Km/h | mmol/l | ml/min/kg | Watt/kg | |
| Maximum | 190 | 19.5 | 7 | 63 | 5.4 | 0 |
| 2 mmol/l | 152 | 15.4 | 2 | 50 | 4.2 | 0 |
| 3 mmol/l | 163 | 16.7 | 3 | 54.1 | 4.5 | 0 |
| 4 mmol/l | 168 | 17.5 | 4 | 56.4 | 4.7 | 0 |
| IAT | 160 | 16.3 | 0 | 52.9 | 4.4 | 0 |

Training Table

| Lactate | HR | v | VO₂ | P | |
|---|---|---|---|---|---|
| mmol/l | 1/min | km/h | ml/min/kg | Watt/kg | |
| 0-1 | 0-125 | 0-11.8 | 0-38.5 | 0-3.1 | REKOM |
| 1-2 | 125-152 | 11.8-15.4 | 38.5-50 | 3.1-4.2 | GA 1 |
| 2-3 | 152-163 | 15.4-16.7 | 50-54.1 | 4.2-4.5 | GA 1-2 |
| 3-4 | 163-168 | 16.7-17.5 | 54.1-56.4 | 4.5-4.7 | GA 2 |
| 4-6 | 168-181 | 17.5-18.7 | 56.4-60.5 | 4.7-5.1 | GA 2 (WSA) |

[image]

1. Use of zeolites for the manufacture of consumption products [dietary supplements] or drugs for lowering the lactate values or the ammonium values in the blood of mammals, including humans.

2. Consumption product or drug according to claim 2, characterized in that the zeolite is chosen from the group of natural zeolites, such as clinoptilolite, silver zeolite, mordenite, phillipsite, and analcite, and/or from the group of the synthetic zeolites such as zeolite A, zeolite W, and zeolite X.

3. Consumption product according to claim 1 or 2, characterized in that it is administered orally.

4. Consumption product or drug according to claim 1 or 2, characterized in that it is administered to adults at a daily dosage of approximately 12 g.

5. Zeolite, characterized in that it is administered for lowering the lactate values or the ammonium values in the blood of mammals, including humans.
